(19) United States
(12) Patent Application Publication
(10) Pub. No.: US 2020/0179247 A1
(43) Pub. Date: Jun. 11, 2020

(54) PERFUMES IN THE FORM OF AQUEOUS MICROEMULSIONS

(71) Applicants: CHANEL PARFUMS BEAUTE, Neuilly Sur Seine (FR); UNIVERSITE DE LILLE, Lille (FR)

(72) Inventors: Oriana BOUCENNA VERDIER, Les Pavillons Sous Bois (FR); Véronique RATAJ-NARDELLO, Pont A Marcq (FR); Jean-Marie AUBRY, Oignies (FR); Grégory DOUYERE, Veneux-Les-Sablons (FR); Corentine MAINGUY, Vannes (FR)

(73) Assignees: CHANEL PARFUMS BEAUTE, Neuilly Sur Seine (FR); UNIVERSITE DE LILLE, Lille (FR)

(21) Appl. No.: 16/618,195

(22) PCT Filed: May 31, 2018
(86) PCT No.: PCT/EP2018/064389

§ 371 Date: Nov. 29, 2019

(30) Foreign Application Priority Data
Jun. 1, 2017 (FR) 1754850

(51) Int. Cl. A61K 8/06; A61K 8/46; A61K 8/60; A61Q 13/00

(52) U.S. Cl. CPC A61K 8/068; A61K 8/463; A61K 8/466; A61K 8/602; A61K 2800/596; A61K 2800/10; A61K 2800/30; A61K 2800/262; A61Q 13/00

(57) ABSTRACT

An oil-in-water microemulsion including, in weight percentages: between 70% and 94% of water; between 1% and 15% of at least one perfumed hydrophobic substance; between 4% and 20% of at least one preferably volatile solvo-surfactant, which is a monoalkylated glycerol derivative of formula (I); and between 0.1% and 15% of at least one anionic surfactant and at least one alkyl glucoside as a hydrotropic agent. The microemulsion can therefore be used to produce a fine fragrance composition or a cosmetic or personal hygiene composition.

The invention relates to aqueous volatile fragrancing microemulsions based on the use of solvo-surfactants.

Perfumes conventionally comprise alcohols such as ethanol or else isopropanol as solvents. However, the use of these solvents has a certain number of disadvantages: they are highly volatile and inflammable, leading to a certain amount of danger when producing them, and to a certain extent when using them. Their own odor may also interfere with that of the perfume. In addition, applied to the skin or the hair, these perfumes may lead to drying out, in particular in consumers with sensitive skin.

Therefore, there is currently an emergence of new fragrancing compositions, especially for reasons of public health and/or ecology. The goal being pursued is that of decreasing or even eliminating volatile organic compounds (such as alcohols) contained in perfumes, by developing fragrancing compositions in the form of stable aqueous dispersions or solutions.

However, the majority of fragrancing molecules are hydrophobic and are therefore not water-soluble. In order to overcome this problem, it is known to use surfactants that make it possible to dissolve fragrancing molecules within micelles, forming microemulsions. It is desirable that the swollen micelles containing the fragrances are small so that the fragrancing composition has a transparent, or at the very least translucent, appearance. Thus, the production of microemulsions meeting this criterion of transparent appearance is of particular benefit.

Other constraints are linked to the thermodynamic stability of the microemulsion, to the non-tacky nature thereof, and to the absence of residue on the skin or on the clothes. It is therefore important to be able to prepare them using as little surfactant as possible.

There is therefore a need for a stable fragrancing composition containing a large amount of fragrance, which is transparent or at least translucent, and which contains as little surfactant as possible.

The aim of the present invention is to provide aqueous, transparent microemulsions that are substantially free of ethanol, containing at least one hydrophobic fragrancing substance (preferably at least 3% and preferentially approximately 10% of fragrance) and at least one volatile solvo-surfactant. Such odoriferous or fragrancing microemulsions are stable and comprises as little as possible of substances that cause undesirable effects, in particular to the skin and/or the environment.

According to the invention, a substance is “volatile” when its boiling point is less than 250° C. at atmospheric pressure. The “non-volatile” compounds have a boiling point of greater than 250° C. at atmospheric pressure.

The present invention therefore relates to a microemulsion of oil-in-water type comprising, preferably consisting of, by weight relative to the total weight of microemulsion:
70% to 94%, preferably 70% to 90%, of water,
1% to 15%, preferably 5% to 12%, of at least one hydrophobic fragrancing substance,
4% to 20%, preferably 4% to 18%, of at least one preferably volatile solvo-surfactant, which is a monoalkylated glycerol derivative of following formula (I):

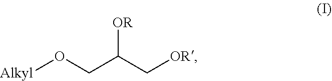

wherein the “alkyl” group is a linear or branched alkyl group comprising from 1 to 8 carbon atoms, preferably from 1 to 5 carbon atoms, and R and R′ are each independently H or a linear or branched alkyl group comprising from 1 to 5 carbon atoms, preferably a methyl or ethyl group, with the proviso that R is different from R′,
0.1% to 15%, preferably 1% to 13%, of at least one anionic surfactant and of at least one alkyl glucoside as hydrotropic agent.

“Microemulsion of oil-in-water type” denotes a liquid system in which an oily (or hydrophobic) phase is dispersed in a continuous aqueous (or hydrophilic) phase so as to form drops of a diameter of less than 100 nm. The oil/water interface is stabilized by surfactant compounds. The drops preferably have a diameter of between 2 and 100 nm.

These microemulsions have drops which are invisible to the naked eye and to optical microscopes. They are transparent, or at the very least translucent, unlike emulsions, which is a desired property especially for fragrancing compositions.

“Hydrophobic substance” denotes a pure substance or a mixture that is water-insoluble or only very sparingly water-soluble by nature. A possible method to determine the hydrophobicity of substances is to measure their solubility in different solvents, or the retention time on a chromatographic column (by high-performance liquid chromatography, HPLC) of said hydrophobic substance.

The hydrophobic substances according to the invention are fragrancing, i.e. they are odoriferous and may be used in perfumes. “Odoriferous substance” is intended to mean a substance that can be olfactorily detected by a subject and/or by olfactometry, according to principles known to those skilled in the art. An example of a method making it possible to detect an odoriferous substance is described in document EP 0003088. Other techniques for detecting an odoriferous substance are applicable, such as gas chromatography techniques, mass spectroscopy techniques or else infrared absorption analysis techniques. Odoriferous substance is also intended to mean a substance which gives off an odor, preferably an odor that is pleasant for at least 20% of people, in particular a fragrance.

The hydrophobic fragrancing substance is preferably a natural or synthetic hydrophobic fragrancing substance, more preferentially natural. It is more preferentially selected from terpenes, essential oils and natural compounds having odoriferous properties (terpenoids), especially selected from aldehydes, esters, ketones, alcohols, phenols, alkenes and ethers.

“Terpenes” denotes hydrocarbons in which the base component is isoprene, their empirical formula comprising a number of carbons that is a multiple of 5, in particular terpenes containing especially 10 or 15 carbon atoms, used in perfumery.

“Terpenoids” denotes terpene derivatives, for example alcohols, phenols, ketones, aldehydes, esters or ethers.

Terpenes and terpenoids are contained in “essential oils”, denoting the concentrated liquid that is commonly odoriferous, volatile, and produced by plants. Essential oils are most commonly extracted from plant organs by hydrodistillation especially, but the constituents of these oils are widely industrially synthesized.

Use may especially be made of the following natural hydrophobic fragrancing substances:
terpenes: pinenes, camphenes, limonene, cadinene, carene, caryophyllene,
alcohols: linalool, geraniol, menthol, citronellol,
ketones: menthone, carvone, beta-ionone, thujone, camphor, cyclopentadecanone,
aldehydes: citral, citrannal, citronellal, cinnamic aldehyde, lilial,
esters: linalyl acetate, menthyl acetate, geranyl acetate, geranyl succinate,
phenols: thymol, carvacrol, eugenol, isoeugenol,
ethers: anethole, eucalyptol, cineole, rose oxide,
alkenes: limonene.

The essential oils may be oils of ylang-ylang, bergamot, eucalyptus, lavender, lavandin, lemongrass, patchouli, peppermint, pine, rose, coriander, Shiu, sage, geranium, palmarosa, Litsea cubeba, lemon, citronnella, orange blossom, grapefruit, lime, mandarin, tangerine, orange, cajeput, camphor, rosemary, green anise, star anise, fennel, basil, tarragon, clove, chilli, thyme, sassafras, wormwood, mugwort, cedar, hyssop, tagetes, rue, elemi, galbanum, juniper berries, cabreuva, lignum vitae, sandalwood, vetiver, ambrette, angelica, iris rhizome, carrot, celery, cumin, lovage, parsley, cinnamon, cardamom, ginger, nutmeg, pepper, frankincense, myrrh, Peru balsam, styrax, buchu, chamomile or rock rose (Jean Garnero, “Huiles essentielles” [Essential oils], Techniques de l'ingénieur, Traité constantes physico-chimiques, K-345).

The amount of hydrophobic fragrancing substances in the microemulsions of the invention is from 1% to 15% by weight, preferably from 5% to 12% by weight relative to the total weight of microemulsion.

“Solvo-surfactant” denotes an amphiphilic compound that brings together some properties of surfactants, especially reduction of water/air surface tension and oil/water interfacial tension, the ability to self-associate in water, and some properties of solvents, especially the ability to evaporate without leaving residues.

Surprisingly, the applicant has discovered and demonstrated that the use of a solvo-surfactant of formula (I) as defined in the present application made it possible to obtain a stable fragrancing composition, which is transparent or at the very least translucent, which comprises a significant amount of fragrance and the least amount of surfactant possible.

According to one advantageous embodiment, the solvo-surfactant present in the microemulsion of the invention is the monoalkylated glycerol derivative of formula (I) as defined above, namely:

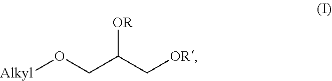

wherein the “alkyl” group is a linear alkyl group comprising 3, 4 or 5 carbon atoms, and R and R′ are each independently H or a methyl or ethyl group, with the proviso that R is different from R′.

The monoalkylated glycerol derivative of formula (I) wherein the “alkyl” group is a linear alkyl group comprising 3 carbon atoms (propyl group), R is H and R′ is methyl, is 1-methoxy-3-propoxypropan-2-ol. It is referred to as “C301” in the examples of the present application.

The monoalkylated glycerol derivative of formula (I) wherein the “alkyl” group is a linear alkyl group comprising 3 carbon atoms (propyl group), R is methyl and R is H, is 2-methoxy-3-propoxypropan-1-ol. It is referred to as “C310” in the examples of the present application.

The monoalkylated glycerol derivative of formula (I) wherein the “alkyl” group is a linear alkyl group comprising 4 carbon atoms (butyl group), R is H and R is methyl, is 1-methoxy-3-butoxypropan-2-ol. It is referred to as “C401” in the examples of the present application.

The monoalkylated glycerol derivative of formula (I) wherein the “alkyl” group is a linear alkyl group comprising 4 carbon atoms (butyl group), R is methyl and R is H, is 2-methoxy-3-butoxypropan-1-ol. It is referred to as “C410” in the examples of the present application.

The monoalkylated glycerol derivative of formula (I) wherein the “alkyl” group is a linear alkyl group comprising 5 carbon atoms (pentyl group), R is H and R is methyl, is 1-methoxy-3-pentoxypropan-2-ol. It is referred to as “C501” in the examples of the present application.

The monoalkylated glycerol derivative of formula (I) wherein the “alkyl” group is a linear alkyl group comprising 5 carbon atoms (pentyl group), R is methyl and R is H, is 2-methoxy-3-pentoxypropan-1-ol. It is referred to as “C510” in the examples of the present application.

Preferably, the solvo-surfactant present in the microemulsion of the invention is the monoalkylated glycerol derivative of formula (I) wherein the “alkyl” group is a linear alkyl group comprising 3, 4 or 5 carbon atoms, R is a methyl group, and R′ is H.

These compounds correspond respectively to the compounds referred to as “C310”, “C410” and “C510” in the examples of the application.

More preferentially, the solvo-surfactant present in the microemulsion of the invention is the monoalkylated glycerol derivative of formula (I) as defined above wherein the “alkyl” group is a linear alkyl group comprising 5 carbon atoms. This compound is 2-methoxy-3-pentoxypropan-1-ol, referred to as “C510”.

The amount of volatile solvo-surfactant in the microemulsion of the ‘invention is from 4% to 20% by weight, preferably from 4% to 18% by weight, and more preferentially still from 4% to 10% by weight.

“Surfactant” denotes a non-volatile compound of amphiphilic nature comprising a polar hydrophilic portion and an apolar hydrophobic portion. A surfactant lowers the surface tension of aqueous solutions and decreases the interfacial tension between water and an immiscible organic liquid. It thus makes it possible to solubilize two immiscible phases, such as water and oil, by interacting with water via its polar portion and with oil via its apolar portion.

The surfactant present in the microemulsion of the invention is an anionic surfactant, namely a surfactant of which the hydrophilic part is negatively charged. Anionic surfactants have proven to be the most effective, within the context of the invention, compared to nonionic, amphoteric or cationic surfactants. “More effective” should be understood to mean, for the purposes of the invention, an introduction of surfactant in a smaller amount in order to form a microemulsion.

According to one advantageous embodiment, the anionic surfactant present in the microemulsion of the invention is selected from:
alkyl sulfonates, and in particular:
sodium C14-17 sulfonate (SAS),
dihexyl sulfosuccinate (DHS) of formula:

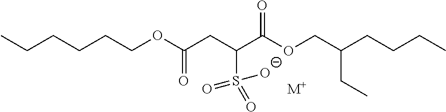

wherein M⁺ represents Na⁺, K⁺, NH₄⁺, (HOCH₂CH₂)₃NH⁺, or
2-ethylhexyl sulfosuccinate (Aerosol OT® or AOT, from CYTEC) of formula:

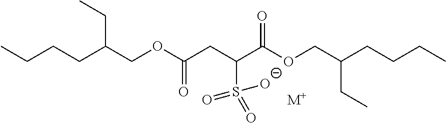

wherein M⁺ represents Na⁺, K⁺, NH₄⁺, (HOCH₂CH₂)₃NH⁺,
alkylaryl sulfonates of formula:

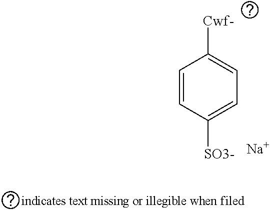

wherein w is an integer from 8 to 12,

and in particular sodium isooctylbenzenesulfonate, sodium isononylbenzenusulfonate or sodium isododecylbenzenesulfonate of formula:

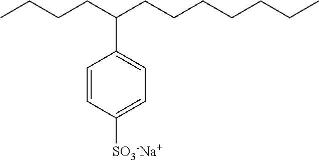

propoxysulfates of formula:

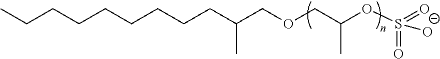

wherein the number of propoxylate units n is from 4 to 8,
alkyl sulfates, especially salts of lauryl sulfate such as sodium lauryl sulfate also known as sodium dodecyl sulfate (SDS), ammonium lauryl sulfate (ALS); sodium alkylether sulfates such as sodium lauryl ether (laureth) sulfate (LES); sodium coco sulfate (SCS),
and salts of fatty acids of formula R—CO₂⁻M⁺, wherein R represents a linear or branched, saturated or unsaturated carbon-based chain containing 8 to 18 carbon atoms, and M⁺ represents a cation selected from the ions Na⁺, K⁺, NH₄⁺, (HOCH₂CH₂)₃NH⁺, especially the oleic acid salt of formula CH₃(CH₂)₇CH═CH(CH₂)₇CO₂⁻M⁺, wherein M⁺ has the above-defined meanings.

The microemulsion comprises an alkyl glucoside as hydrotropic agent. “Hydrotropic agent” is intended to mean an amphiphilic compound comprising hydrophilic functional groups, used to enable the solubilization of poorly soluble substances in an aqueous solution.

Surprisingly, the applicant has demonstrated that the combination of at least one anionic surfactant and of at least one alkyl glucoside makes it possible to increase the cloud point of the microemulsion.

The “cloud point” according to the invention is understood to mean the temperature starting from which the microemulsion changes from a transparent (or even translucent) state to a cloudy, or even milky, state. The microemulsion according to the invention must retain a stability under conventional usage conditions, and in fact must be stable, and therefore remain transparent and even translucent, in a range of temperatures between 5° C. and 45° C. inclusive.

Thus, by means of a well-judged combination of surfactant and alkyl glucoside, it is possible to obtain a stable microemulsion using a very small amount of surfactant. Microemulsions containing only the surfactant alone or containing only the alkyl glucoside alone, in equivalent or quasi-equivalent amounts are not, on the other hand, stable. There is therefore a synergy effect between the anionic surfactant and the alkyl glucoside.

Moreover, this synergy effect also has a positive impact on the amount of solvo-surfactant introduced into the microemulsion. Thus, by means of the combined use of anionic surfactant and alkyl glucoside, it is possible to use smaller amounts of solvo-surfactant, namely ranging from 4% to 10% by weight, relative to the total weight of the microemulsion. In the examples below, an amount of 5% of solvo-surfactant makes it possible to obtain a stable microemulsion and the perfect solubilization of the hydrophobic fragrancing substance. The reduction in the amount of solvo-surfactant takes place in favor of the amount of water which may thus be increased. The aqueous nature of the microemulsion is thus increased.

According to one advantageous embodiment, the alkyl glucoside present in the microemulsion of the invention is selected from heptyl glucoside, octyl glucoside, decyl glucoside and mixtures thereof, and is preferably heptyl glucoside.

The amount of anionic surfactant and of alkyl glucoside present in the microemulsion of the invention is from 0.1% to 15% by weight, preferably from 1% to 13% by weight relative to the total weight of microemulsion.

More particularly, in the microemulsion of the invention:
the amount of anionic surfactant is less than or equal to 2% by weight relative to the total weight of microemulsion,
the amount of alkyl glucoside is from 0.5% to 13% by weight relative to the total weight of microemulsion, and preferably from 0.5% to 8%.

According to one advantageous embodiment of the invention, the anionic surfactant:alkyl glucoside ratio is from 1:4 to 1:12, and preferably from 1:4 to 1:10.

The microemulsion according to the invention preferably consists of:
70% to 90% of water,
5% to 12% of at least one hydrophobic fragrancing substance,
4% to 18% of at least one volatile solvo-surfactant,
1% to 13% of at least one anionic surfactant and of at least one alkyl glucoside as hydrotropic agent.

The microemulsion according to the invention is preferably substantially free of ethanol, i.e. it comprises less than 3% by weight of ethanol, preferably less than 2% by weight of ethanol, preferably less than 1% by weight of ethanol. More preferentially, it is devoid of ethanol, i.e. it contains 0% by weight of ethanol.

The microemulsion according to the invention makes it possible in particular to obtain an aqueous fragrancing composition having a transparency similar to an alcoholic product, without a greasy or tacky feel, and does not leave traces or residues on the fabrics or the skin.

Optionally, the microemulsion according to the invention may comprise from 0.01% to 2% by weight of at least one additive.

According to a preferred embodiment, the microemulsion according to the invention may comprise, nonlimitingly, one or more of the following additives:
an antifoaming agent selected from decamethylcyclopentasiloxane, dimethicone, cetyl dimethicone, dimethicone copolyol, dimethiconol, hexamethyldisiloxane, hexamidine diisethionate, hexyl alcohol, hexyldeceth-2, isopropyl alcohol, phenethyl disiloxane, phenyl trimethicone, polysilicone-10, polysilicone-7, polysilicone-8, propyl alcohol, silica dimethyl silylate, silica silylate, simethicone, tetramethyl decynediol and trimethylsiloxysilicate;
a preservative selected for example from sodium benzoate, potassium benzoate, sodium salicylate, caprylyl glycol, pentylene glycol, ethylhexylglycerin, chlorphenesin, chlorhexidine, chlorhexidine digluconate, methylisothiazolinone, methylparaben, propylparaben, phenethyl alcohol and phenoxyethanol;
an antioxidant selected for example from BHA, BHT and tocopherol.

The microemulsions according to the invention have a fragrance quality that is stable over time, corresponding to the standard shelf life of a cosmetic product, and stable at temperatures from 5 to 45° C., corresponding to the temperatures of exposure and use of a cosmetic product.

The technical criteria of the quality of a fragrance, in the case of an aqueous fragrance, are:
the ability of a fragranced composition to maintain, after application, an olfactory perception threshold over time,
the ability of a composition, once applied, to maintain its olfactory form over time,
the ability of a composition to not undergo endogenous or exogenous alterations that could modify its olfactory form, and
harmlessness, which is the ability of a composition to not produce undesirable effects once it is applied to the skin of the user.

Highly advantageously, the microemulsions of the invention are thermodynamically stable and have a transparent or translucent appearance and, even more advantageously, the microemulsions of the invention are thermodynamically stable and have a transparent or translucent appearance for at least one or two years.

The microemulsions of the invention are advantageously used for the preparation of compositions applied in:
fine fragrance, or
cosmetics and body hygiene products.

Thus, the present invention also relates to the use of a microemulsion according to the invention, for the preparation of a fine fragrance composition, or of a cosmetic or body hygiene composition.

The microemulsions may be used in cosmetics. They may then contain in particular, and without this list being limiting, one or more compounds selected from silicones, paraffin oil, isooctane, isodecane, squalene, squalane, sebum and lanolin.

In the following examples, the names, abbreviations and structures of the solvo-surfactants tested, namely monomethylated glycerol derivatives (I), are the following:

| NAMES | ABBREVIATIONS | STRUCTURES |
|---|---|---|
| Monomethylated propylglycerol | C301 | 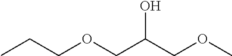 |
| Monomethylated propylglycerol | C310 | 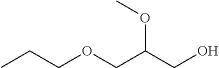 |
| Monomethylated butylglycerol | C401 | 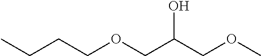 |
| Monomethylated butylglycerol | C410 | 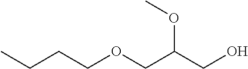 |
| Monomethylated pentylglycerol | C501 | 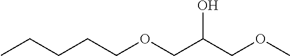 |
| Monomethylated pentylglycerol | C510 | 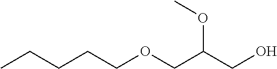 |

### EXAMPLE 1: SYNTHESIS OF THE SOLVO-SURFACTANTS ACCORDING TO THE INVENTION, THE MONOMETHYLATED GLYCEROL DERIVATIVES OF FORMULA (I)

The syntheses of 1-methoxy-3-propoxypropan-2-ol (C301) and 1-methoxy-3-pentoxypropan-2-ol (C501) are carried out in two steps. The synthesis of 1-methoxy-3-butoxypropan-2-ol (C401) is carried out simply by opening the epoxide, insofar as the starting reagent, butylglycidol, is commercially available.

a) Condensation of the Alcohol to Epichlorohydrin

The condensation of the alcohol (1 mol) to epichlorohydrin is carried out with a slight excess of epichlorohydrin (1.5 mol) in the presence of ZnCl₂ as catalyst. The epichlorohydrin is added dropwise for 1 h at 100° C. The reaction medium is then kept at 115° C. for 5 h then cooled to 50° C. NaOH (2.3 moles) at 48% is then added dropwise for 1 h. All these synthesis steps are carried out with vigorous stirring. Once the reaction has ended, distilled water is added and the product is then washed twice with water to eliminate residual salts.

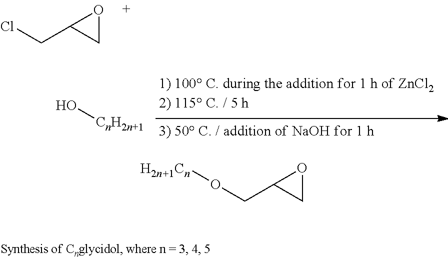

The product obtained is finally distilled under vacuum at 10-20 mbar between 75 and 80° C.

b) Opening of the Epoxide with Methanolate

The Cₙglycidol (where n=3, 4, 5) is added dropwise to a solution of methanol containing sodium methanolate obtained beforehand by reaction between methanol and solid sodium. The addition is carried out at reflux at 80° C. for 30 min, then the temperature of 80° C. is maintained for 24 h until the Cₙglycidol disappears entirely:

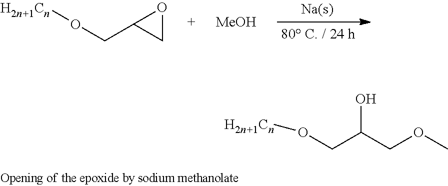

Purification:

The methanol is evaporated on a rotary evaporator once the reaction has ended. The product obtained is then washed with two aqueous solutions saturated with NaCl: one containing 3.4% HCl and the other containing 10% NaHCO₃.

c) Synthesis of the Compounds C310, C410, and C510

The synthesis of the compounds C310, C410 and C510 is carried out in three steps starting from Cₙglycidol (where n=3, 4, 5) as depicted in scheme 1 below.

The first step is carried out under the conditions of step b) described above, replacing the methanol with benzyl alcohol. In a second step, the intermediate thus obtained is reacted in a basic medium in the presence of dimethyl sulfate in order to obtain the methylation of the free secondary alcohol function. After a third step of catalytic hydrogenation in the presence of hydrogen and palladium-on-carbon, the final product is obtained.

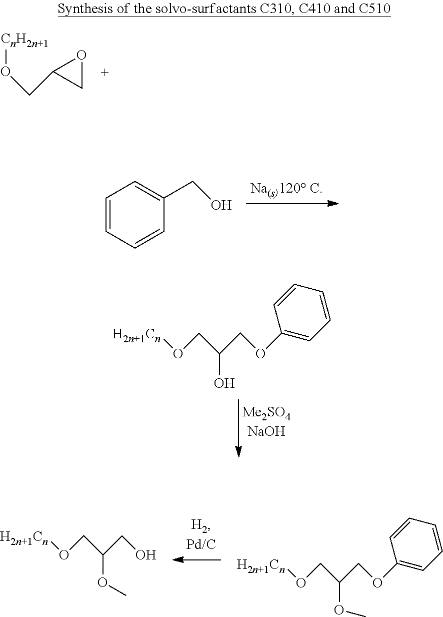

### EXAMPLE 2: SOLUBILIZATION OF FRAGRANCE CONCENTRATES

Solubilization by Solvo-Surfactants Alone

Solubilization of the Gamma-Undecalactone Fragrance

The aim of this test is to classify the solvo-surfactants according to their capacity to solubilize (or not) gamma-undecalactone.

Formulae containing 5% of gamma-undecalactone, 4.4% of SDS (anionic surfactant), X % of solvo-surfactant and QS of water were produced.

The table below describes in detail the amount of SS needed to solubilize the fragrance.

| C510 | C501 | C401 | C410 | C310 |
|---|---|---|---|---|
| 9.1% | 9.3% | 12.2% | 16.2% | 20% |

The following classification is obtained: C510>C501>C401>C410>C310.

The solvo-surfactant having the best capacities to solubilize the hydrophobic fragrancing substance is C510.

### EXAMPLE 3: TEMPERATURE STABILITY OF FRAGRANCING AQUEOUS MICROEMULSIONS

The following perfume extract (P) was prepared:

| Name of fragrance | CAS # | Quantity (%) |
|---|---|---|
| GAMMA-UNDECALACTONE | 104-67-6 | 0.62 |
| CIS-3-HEXENYL ACETATE | 3681-71-8 | 1.21 |
| HEXENYL CIS-3-BENZOATE | 25152-85-6 | 2.42 |
| EUGENOL | 97-53-0 | 2.42 |
| BETA-IONONE | 14901-07-6 | 3.64 |
| GAMMA-METHYLIONONE | 127-51-5 | 6.06 |
| BENZYL PROPIONATE | 122-63-4 | 1.21 |
| BENZYL ACETATE | 140-11-4 | 6.06 |
| HEDIONE HC | 24851-98-7 | 36.36 |
| ISO GAMMA SUPER | 68155-66-8 | 31.52 |
| CIS-3-HEXENYL SALICYLATE | 65405-77-8 | 6.06 |
| VANILLIN | 121-33-5 | 2.42 |

Then, the following formulations were prepared:

| | | | | | Alkyl | |
|---|---|---|---|---|---|---|
| | | Fra- | Solvo- | | glucoside | Stability |
| Formu- | | grance | surfactant | Surfactant | (Heptyl- | 5° C. to |
| lation | Water | (P) | (C510) | (SDS) | glucoside) | 45° C. |
| A | 80.4% | 5% | 12.5% | 2.1% | NO | KO |
| B | 78.9% | 5% | 15% | NO | 1.1% | KO |
| C | 76.7% | 5% | 15% | 2.0% | 1.3% | OK |
| D | 85.34% | 5% | 5% | 0.66% | 4.0% | OK |
| E | 85.0% | 5% | 5% | NO | 5.0% | KO |

In order to be acceptable, the formulations A, B, C, D and E must be temperature stable over a range extending from 5° C. to 45° C.

The formulations are therefore placed in a bath at 5° C. and at 45° C. for 24 h.

After observation, the clear formulations are considered to be stable and the cloudy formulations are considered to be unstable. The table above gives the stable compositions (“OK”) and the compositions that led to destabilization (appearance of cloudiness) (“KO”).

Only the formulations C and D comprise a combination of an anionic surfactant and an alkyl glucoside having a stability over the whole of the 5° C. to 45° C. temperature range.

Advantageously, composition D has all of the benefits of the invention owing to the use of a well-judged “surfactant:alkyl glucoside” ratio which is 1:6, making possible to obtain an aqueous formulation rich in water (>85%) in the presence of a small amount of solvo-surfactant (5%).

### EXAMPLE 4: COMPARATIVE TEST

A microemulsion of the invention, namely the formulation D described in the preceding example 3, which comprises the solvo-surfactant C510, is compared to two other microemulsions (formulations D1 and D2) which respectively comprise hexylene glycol and propylene glycol instead of the solvo-surfactant C510.

The hexylene glycol and propylene glycol do not correspond to the formula (I) of the solvo-surfactant of the invention.

| | D | | |
|---|---|---|---|
| Formulation | (invention) | D1 | D2 |
| Water | 85.34% | 85.34% | 85.34% |
| Fragrance (P) | 5% | 5% | 5% |
| SDS | 0.66% | 0.66% | 0.66% |
| Heptyl glucoside | 4% | 4% | 4% |
| C510 | 5% | — | — |
| Hexylene glycol | — | 5% | — |
| Propylene glycol | — | — | 5% |

The perfume extract P is as described in example 3.

The heptyl glucoside used is the one sold by SEPPIC under the name “Sepiclear G7”.

The sodium lauryl sulfate (SDS) used is the one sold by BASF under the name “Texaplon LS30”.

As indicated, in order to be acceptable, the formulations D, D1 and D2 must be temperature stable over a range extending from 5° C. to 45° C.

The formulations are placed in a bath at 5° C. and at 45° C., for 24 h.

After observation, the clear formulations are considered to be stable and the cloudy formulations are considered to be unstable.

It is thus observed that at D0, at room temperature, the formulation D is clear whilst the formulations D1 and D2 are already opalescent.

At the end of 24 hours, both at 5° C. and at 45° C., the formulations D1 and D2 are cloudy and a significant phase separation is observed, whilst the formulation D of the invention has remained clear.

This comparative test therefore demonstrates the importance played by the solvo-surfactant of the invention.

1-11. (canceled)

12. A microemulsion of oil-in-water type comprising by weight relative to the total weight of microemulsion:
70% to 94%, preferably 70% to 90%, of water,
1% to 15%, preferably 5% to 12%, of at least one hydrophobic fragrancing substance,
4% to 20%, preferably 4% to 18%, of at least one preferably volatile solvo-surfactant, which is a monoalkylated glycerol derivative of following formula (I): 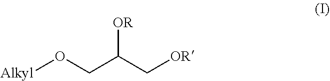
wherein the alkyl group is a linear or branched alkyl group comprising from 1 to 8 carbon atoms, preferably from 1 to 5 carbon atoms, and R and R′ are each independently H or a linear or branched alkyl group comprising from 1 to 5 carbon atoms, preferably a methyl or ethyl group, with the proviso that R is different from R′,
0.1% to 15%, preferably 1% to 13%, of at least one anionic surfactant and of at least one alkyl glucoside as hydrotropic agent.

13. The microemulsion as claimed in claim 12, wherein the hydrophobic fragrancing substance is a natural hydrophobic fragrancing substance selected from terpenes, essential oils and natural compounds having odoriferous properties, especially selected from aldehydes, esters, ketones, alcohols, phenols, alkenes and ethers.

14. The microemulsion as claimed in claim 12, wherein the solvo-surfactant is the monoalkylated glycerol derivative of formula (I) wherein the alkyl group is a linear alkyl group comprising 3, 4 or 5 carbon atoms, R is a methyl group and R′ is H.

15. The microemulsion as claimed in claim 14, wherein the solvo-surfactant is the monoalkylated glycerol derivative of formula (I) wherein the alkyl group is a linear alkyl group comprising 5 carbon atoms.

16. The microemulsion as claimed in claim 12, wherein the anionic surfactant is selected from:
(i) alkyl sulfonates, and in particular:
sodium C14-17 sulfonate (SAS),
dihexyl sulfosuccinate (DHS) of formula: 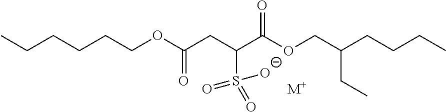
wherein M+ represents Na⁺, K⁺, NH₄⁺, (HOCH₂CH₂)₃NH⁺, or 2-ethylhexyl sulfosuccinate of formula: 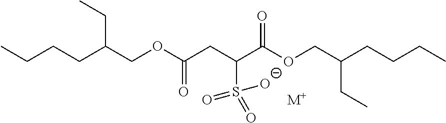
wherein M⁺ represents Na⁺, K⁺, NH₄⁺, (HOCH₂CH₂)₃NH⁺,
(ii) alkylaryl sulfonates of formula: 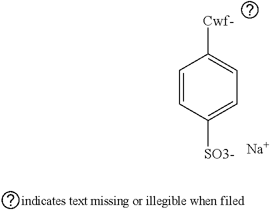
wherein w is an integer from 8 to 12,
and in particular sodium isooctylbenzenesulfonate, sodium isononylbenzenesulfonate or sodium isododecylbenzenesulfonate of formula: 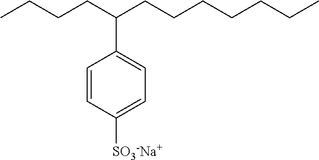
(iii) propoxy sulfates of formula: 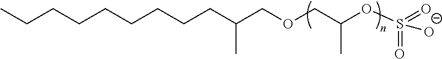
wherein the number of propoxylate units n is from 4 to 8,
(iv) alkyl sulfates, especially salts of lauryl sulfate such as sodium lauryl sulfate also known as sodium dodecyl sulfate (SDS), ammonium lauryl sulfate (ALS); sodium alkylether sulfates such as sodium lauryl ether (laureth) sulfate (LES); sodium coco sulfate (SCS),
(v) and salts of fatty acids of formula R—CO₂⁻M⁺, wherein R represents a linear or branched, saturated or unsaturated carbon-based chain containing 8 to 18 carbon atoms, and M⁺ represents a cation selected from the ions Na⁺, K⁺, NH₄⁺, (HOCH₂CH₂)₃NH⁺, especially the oleic acid salt of formula CH₃(CH₂)₇CH═CH(CH₂)₇CO₂⁻M⁺, wherein M⁺ has the above-defined meanings.

17. The microemulsion as claimed in claim 12, wherein the alkyl glucoside is in particular selected from heptyl glucoside, octyl glucoside, decyl glucoside and mixtures thereof, and is preferably heptyl glucoside.

18. The microemulsion as claimed in claim 12, wherein:
the amount of anionic surfactant is less than or equal to 2% by weight relative to the total weight of microemulsion,
the amount of alkyl glucoside is from 0.5% to 13% by weight relative to the total weight of microemulsion, and preferably from 0.5% to 8%.

19. The microemulsion as claimed in claim 12, wherein the anionic surfactant:alkyl glucoside ratio is from 1:4 to 1:12, and preferably from 1:4 to 1:10.

20. The microemulsion as claimed in claim 12, wherein the amount of solvo-surfactant is from 4% to 10% by weight relative to the total weight of microemulsion.

21. The microemulsion as claimed in claim 12, which is substantially free of ethanol, preferably which is devoid of ethanol.

22. A fine fragrance composition or of a cosmetic or body hygiene composition comprising the microemulsion as claimed in claim 12.
